(19) United States
(12) Patent Application Publication
(10) Pub. No.: US 2013/0165810 A1
(43) Pub. Date: Jun. 27, 2013

(54) RESPIRATION MONITORING DEVICE

(71) Applicants: Reza Saatchi, Sheffield (GB); Stuart McGrath, Hyde (GB)

(72) Inventors: Reza Saatchi, Sheffield (GB); Stuart McGrath, Hyde (GB)

(73) Assignee: SHEFFIELD HALLAM UNIVERSITY, Sheffield (GB)

(21) Appl. No.: 13/699,985

(22) PCT Filed: May 19, 2011
(86) PCT No.: PCT/GB2011/050951

§ 371 Date: Feb. 20, 2013

(30) Foreign Application Priority Data
May 24, 2010 (GB) 1008591.8

(51) Int. Cl. A61B 5/087; A61B 5/00; A61B 5/097

(52) U.S. Cl. CPC A61B 5/0878; A61B 5/097; A61B 5/742; A61B 5/7475; A61B 5/0004; A61B 5/7225
USPC 600/537

(57) ABSTRACT

A respiration monitoring device configured to provide quantitative data output of respiration cycles of a human or animal. The device may be configured to output a respiration rate and/or a respiration waveform expressed as temperature verses time. The device comprises a temperature modifier to heat or cool a flow of exhaled air. This heated or cooled air then flows past a temperature sensor to determine a temperature change associated with each exhaled air cycle.

The present invention relates to apparatus and method for monitoring respiration of a human or animal using an electronic temperature sensor to detect a temperature change resultant from of a flow of exhaled air that has been heated or cooled by a temperature modifier.

Respiratory rate is an important physiological measure used in clinical and sports environments to examine the health or performance of an individual. This measurement is even more important in vulnerable patients, for example the critically ill, neonates, infants and the elderly.

Respiration monitoring can be contact or noncontact based. In contact based respiration monitoring approaches, the sensing device is attached to the subject's body. A widely used contact based respiration monitoring method relies on thermistors being placed close to nostrils to detect the temperature of exhaled and inhaled air. Another contact approach involves the use of strain-gage pressure sensors incorporated in a strap to detect chest and abdominal movements. A number of studies reported extraction of respiration signal from an electrocardiogram (B. Mazzanti, et al, “validation of an ECG-derived respiration monitoring method”, Computers in Cardiology, vol. 30, pp. 613-616, 2003.); (S. Park, et al, “an improved algorithm for respiration signal extraction from electrocardiogram measured by conductive textile electrodes using instantaneous frequency estimation”, Medical & Biological Engineering & Computing, vol. 46, no. 2, pp. 147-158, 2008). These contact approaches have significant drawbacks. For example the attachment of the sensors to the patient's body causes discomfort and the resulting stress can affect breathing rate. The contact based thermistor approach has a further disadvantage as the sensing device needs to be disposed after a single use for hygiene reasons.

Respiration monitoring based on audio sensing can be either contact or non-contact. In non-contact audio based respiration monitoring, the sensor is often required to be placed very close to the patient and therefore the technique suffers from the same drawbacks as contact-based solutions (P. Corbishley et al, “breathing detection: towards a miniaturized, wearable, battery-operated monitoring system”, IEEE Transactions on Biomedical Engineering, vol. 55, no. 1, pp. 196-204, 2001.) (R. Jané, et al, “automatic detection of snoring signals: validation with simple snorers and OSAS patients”, Proc. of the 22nd Annual International Conference of the IEEE Engineering in Medicine and Biology Society. pp. 3129-3131, 2000).

A number of non-contact respiration monitoring systems have also been reported. These systems included human breath temperature measurements using infrared sensing devices (Z. Zhu, et al, “tracking human breath in infrared imaging”, in Proceedings of the fifth Symposium on Bioinformatics and Bioengineering, IEEE Computer Society Washington, D.C., USA, pp. 227-231, 2005.) or by measuring the CO₂ in exhaled air (R. Murthy, et al, “touchless monitoring of breathing function”, Engineering in Medicine and Biology Society, 2004. IEMBS'04 26th Annual International Conference of the IEEE, vol. 1, 2004.); (J. Fei, et al, “imaging breathing rate in CO₂ absorption band”, Proceedings of the 2005 IEEE Engineering in Medicine and Biology 27th Annual Conference, Shanghai, China, pp. 700-705, 2005). Vision based respiration monitoring is another approach (I. Sato et al, “non-contact Breath Motion Monitoring System in Full Automation”, Proceedings of the 2005 IEEE Engineering in Medicine and Biology 27th Annual Conference, Shanghai, China, pp. 3448-3451, 2005.); (M. Frigola, et al, “vision Based Respiratory Monitoring System”, Proceedings of the 10th Mediterranean Conference on Control and Automation—MED2002 Lisbon, Portugal, Jul. 9-12, 2002.) (C. W. Wang, et al, “vision analysis in detecting abnormal breathing activity in application to diagnosis of obstructive sleep apnea”, Proceedings of the 28th IEEE EMBS Annual International Conference, New York City, USA, pp. 4469-4473, 2006). This approach relies on video recording of the chest and abdominal movements.

However, current non-contact respiration monitoring systems are disadvantageous due largely to their sophistication (and hence high cost) and the level of skill needed to operate them. They are also of very limited use in environments such as outpatients and in ambulances (because they require extensive instrumentation set up). In many cases, respiration is clinically monitored by the medical staff placing the back of their hand in close proximity to the mouth and nostrils so as to sense the cycles of exhaled air and to then determine manually the respiration rate or by visually counting the chest movements produced by respiration.

What is required is apparatus and method enabling the quantitative assessment of respiration that is convenient to operate and is suitable for widespread use, both by trained and non-trained medical staff, and in a variety of sport-related monitoring activities.

Accordingly, the inventors provide a respiratory monitoring device primarily designed to be non-contact, but also configurable for positioning at or attaching to a subject. The device is configured to output quantitative data on the respiratory cycle, possibly expressed as breaths or cycles per minute and/or a graphical waveform expressed as temperature verses time. By evaluating the peak-to-peak distance of the waveform it is then possible to calculate the respiration rate as cycles per minute. The graphical waveform is also useful to assist with the assessment of the health of a subject by analysis of the shape of the waveform in the time domain or by analysing its frequency spectrum through methods such as Fourier analysis or wavelet transform.

According to a preferred embodiment, temperature sensing is provided by a thermistor configured to receive an airflow resultant from air exhaled from the lungs of a subject. The sensitivity of the present device, and therefore accuracy of the results, is achieved by modifying the airflow temperature flowing through the device using a temperature modifier acting to either heat or cool the air at a position upstream of the temperature sensor.

According to a first aspect of the present invention there is provided a respiration monitoring device comprising: an airflow inlet port to allow a flow of exhaled air from a human or animal into the device; a temperature modifier to receive the flow of exhaled air via the airflow inlet port and to heat the exhaled air; an electronic temperature sensor to detect a change in temperature of air within the device; an airflow tunnel configured to direct the flow of exhaled air from the airflow inlet port to the temperature sensor, the temperature modifier positioned at and external to the tunnel so as to heat the exhaled air as it flows through the tunnel; wherein the temperature sensor is positioned external to an open airflow exit end of the tunnel in an airflow path exiting the tunnel downstream of the temperature modifier to receive the heated flow of air from the tunnel.

Preferably, the electronic temperature sensor comprises a thermistor such as an NTC or PTC thermistor. Preferably, the temperature modifier comprises a PTC heater. Preferably, the tunnel comprises a metal and in particular a steel tunnel.

The sensitivity of the device may be changed by altering the type of temperature sensing device or the gain of an operational amplifier used to amplify the signal from the temperature sensing device. The shape and size of the air funnel (attached to the air inlet port) that guides the exhaled air into the air tunnel also affect sensitivity and may be provided in a variety of forms.

Preferably, the device further comprises an air funnel extending from the airflow inlet port to direct the flow of exhaled air into the device. Preferably, means for releasably attaching the funnel to the device are provided in the form of snap click, bayonet or tongue and groove type connections.

The device may comprise at least one airflow outlet port to allow the flow of exhaled air to exit the device once it has passed the temperature sensor.

Preferably, the device further comprises an electronic display screen and in particular an LCD or LED display screen. Optionally, the displace screen may comprise a mechanical key pad or a touch screen device having keypad functionality forming part of the screen.

According to specific implementations, the device may comprise a microchip, an electronic memory for data storage, electronic communication means to enable wired or wireless information transfer from the device, an analogue to digital convertor, a microprocessor and at least one battery power source to provide power to the internal electronic components and/or PCBs within the device.

According to a specific implementation a user interface at the device or a remote PC is created using LabVIEW. According to further embodiments, the user interface is created by custom written code (for example visual C). Alternatively, the present apparatus and method is suitable for use by accommodating any existing commercial interface that may be adapted to suit the requirements of a particular application (for example medical or sport) and to output the respiration rate data as a graphical waveform and/or numeric data.

Preferably, the device comprises and is implemented with a warning threshold to alert a user of the device when a patient's respiration falls outside a predefined range. This range input means is variable to suit different patients such as infants, children and adults. The warning threshold feature may be optional and can be manually or automatically adjusted by a user via the user interface and keypad features on the device and/or remotely via a PC wired or in wireless communication with the handheld device.

Optionally, the respiration data are transferred in real-time to a remote PC via wired or wireless communication. Alternatively, data may be stored at the device via suitable memory and processed at the device via a suitable processor for output at a visual display. Processing includes for example, determining the average respiration rate and its standard deviation, maximum and minimum respiration rate over a specific time interval. The on-device memory storage facility may also be configured to allow input and storage of a patient's details (including name, date of birth, time of data recording etc). This data may be entered via a PC and transmitted via wired or wireless communication device or input directly at the device via an on-device keypad or touch screen. Respiration data acquired from the patient would then be stored within a patient's file on the device for output at the device or remotely via a PC.

According to a specific implementation, the handheld device may comprise a printer configured to output a hardcopy of the raw respiration data or processed data in the form of a waveform and/or numeric values. Alternatively, the device may provide output via wired or wireless communication to a remote PC and/or printer.

The user interface and software of the device may be configured to provide detailed analysis of the respiration signal obtained from the patient so as to identify specific respiration patterns. For example, the respiration rate as determined may be within an acceptable range, however the respiration pattern (output waveform) may be unsteady (manifested as slow and fast respiration cycles) possibly indicating a particular physiological condition. The supporting software may be configured to identify such anomalies automatically and/or allow a user to undertake a detailed analysis of the waveform pattern and/or statistical parameters calculated by the software to assess a patient's health and/or sporting performance for example.

In one embodiment, the device comprises a rechargeable battery accommodated at the handheld device. A suitable port or docking station is provided to interface with a recharging station which is in turn connected to the mains electricity. The handheld device may therefore be conveniently recharged ready for subsequent use. Furthermore, the device may comprise suitable electronics and/or software to provide an automatic power-off after a predetermined time has elapsed of device inactivity. This way, the device is configured to be energy saving. This automatic power-off feature may be touch sensitive such that when the device is held by a user, it is automatically on and automatically shuts-down when left standing between use periods.

The device may also comprise a clip, strap or suitable attachment means to enable the portable device to be attached to the clothing of a person, or conveniently carried by a person when not in use. Similarly, the device may comprise a removable protective outer jacket, preferably of rubber, to protect the device against damage if accidently dropped. The protective jacket may also be waterproof to protect the internal electronics. The outer jacket is configured to enable the device to be used whilst being protected and comprises suitable openings for the keypad/touch screen and other ports for connection to peripheral devices and networking. The device may also comprise a clock and time display feature implemented via a common display screen used to display the respiration data or a separate display screen.

According to a second aspect of the present invention there is provided a method of monitoring respiration of a human or animal comprising: receiving a flow of exhaled air via an airflow inlet port; directing the flow of exhaled air within the device through a tunnel, heating the flow of exhaled air received from the air flow inlet port within the tunnel using a temperature modifier, the temperature modifier positioned at and external to the tunnel so as to heat the exhaled air as it flows through the tunnel; detecting a change in temperature of air within the device using an electronic temperature sensor positioned external to an open airflow end of the tunnel end at one end of the tunnel in an airflow path exiting the tunnel downstream of the temperature modifier as the heated exhaled air flows from the open end of the tunnel.

A specific implementation of the present invention will now be described, by way of example only and with reference to the accompanying drawings in which:

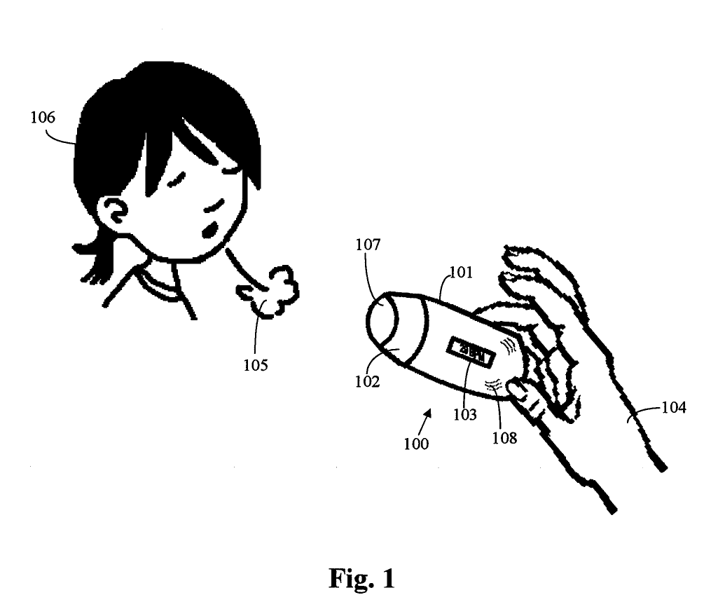

FIG. 1 illustrates schematically a handheld respiration monitoring device to monitor respiration of a child according to a specific implementation of the present invention;

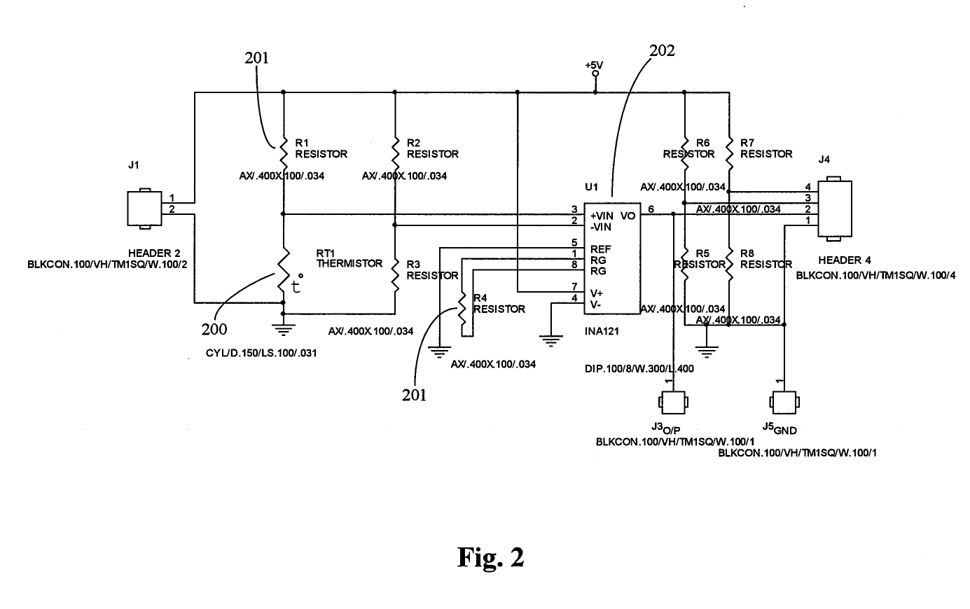

FIG. 2 illustrates schematically an electronic circuit diagram of a measurement part of a temperature sensor circuit implemented as a thermistor according to a specific implementation of the present invention;

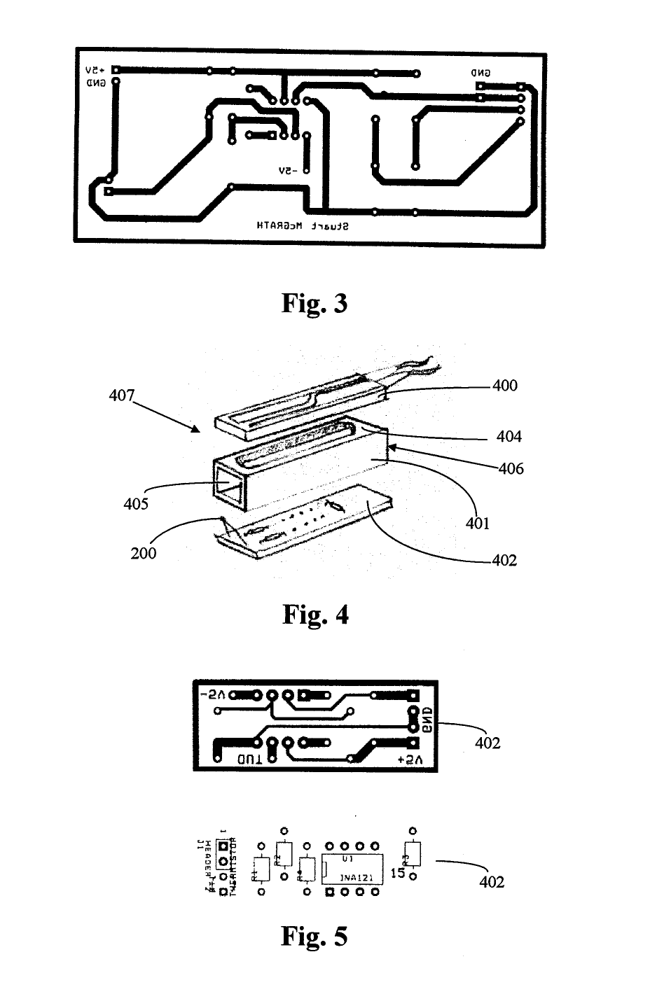

FIG. 3 illustrates a printed circuit board design for the circuit diagram of FIG. 2;

FIG. 4 illustrates an exploded view of selected components of a respiration monitoring device having a PCT heater, a metal air flow tunnel and printed circuit board comprising thermistor according to a specific implementation of the present invention;

FIG. 5 illustrates a reduced component printed circuit board according to that of FIG. 3;

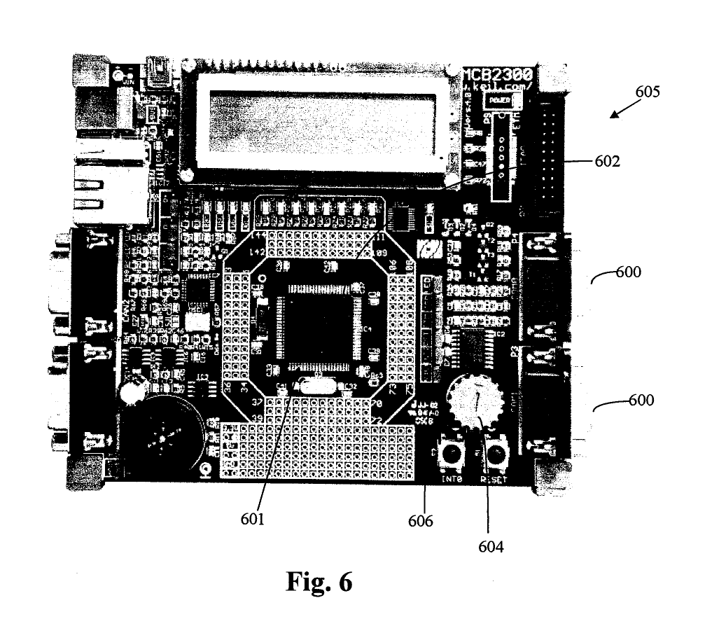

FIG. 6 illustrates an electronics evaluation board comprising a microcontroller and an LCD, the evaluation board configured for interfacing with the temperature sensor to monitor respiration cycles according to a specific implantation of the present invention;

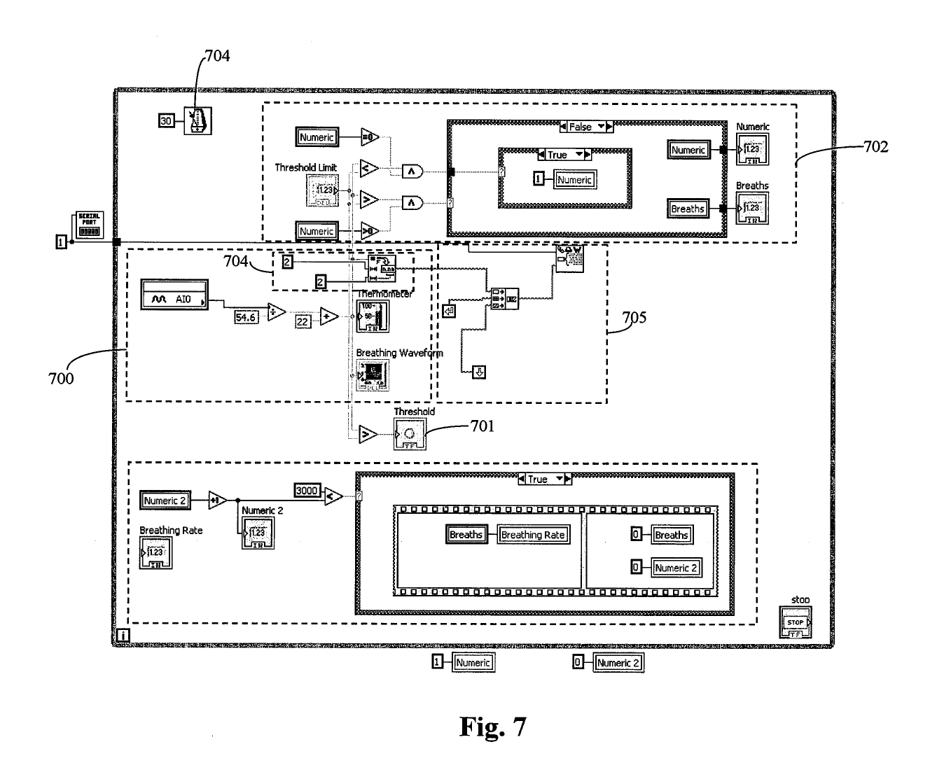

FIG. 7 illustrates schematically a user interface for data manipulation and analysis to obtain a respiration rate for a human or animal according to a specific implementation of the present invention;

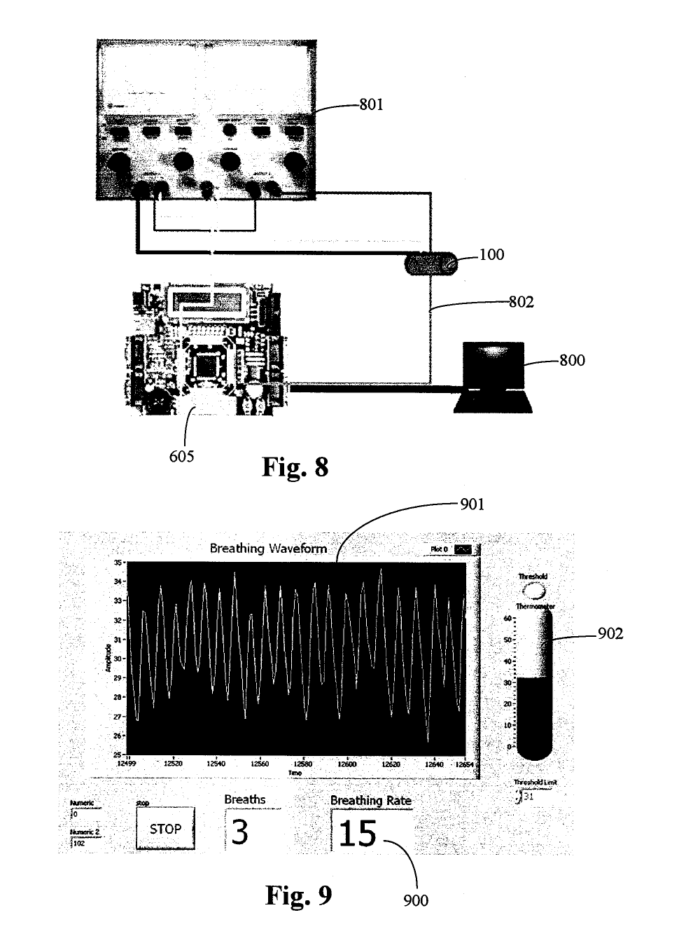

FIG. 8 illustrates an experimental testing apparatus having a dual power supply, a microcontroller evaluation board, a breathing monitor sensor and heater circuit coupled to a PC according to a specific implementation of the present invention;

FIG. 9 illustrates an output display and user interface for the data obtained from the temperature sensor to determine a breathing rate and breathing wave form pattern;

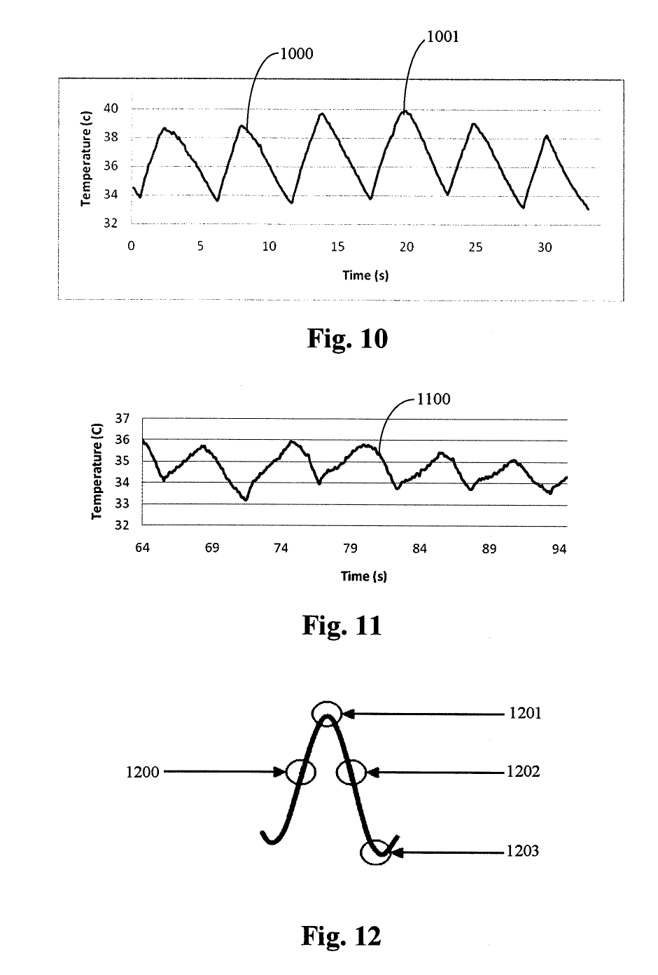

FIG. 10 is a respiratory cycle waveform generated by test subject A expressed as temperature over time;

FIG. 11 is a respiratory cycle waveform generated by test subject B expressed as temperature over time; and

FIG. 12 illustrates schematically the characteristics of an ideal respiratory cycle waveform expressed as temperature over time.

The respiration monitoring device 100 comprises a body 101 to house the various electronic components, including in particular the temperature modifier and the electronic temperature sensor. Body 101 comprises a suitable hollow plastic case having an airflow inlet port 107 to allow a flow of exhaled air into device 100. Inlet port 107 is formed as a funnel or shroud 102 extending from main body 101 and configured to channel the flow of exhaled air 105 into the device 100. Airflow outlet ports 108 are provided at body 101 to allow the exhaled air 105 flowing through device 100 to exit main body 101.

The respiration monitor 100 is suitably sized so as to be grasped between the figures and thumb 104 of a user so as to provide non-contact respiration monitoring by being held in close proximity to a human or animal and in particular a human infant or child 106.

A visual display 103 is mounted at main body 101 to output visually the quantitative results of respiration monitoring, for example expressed as breaths or cycles per minute.

According to further specific implementations display device 103 may comprise a touch screen display, including an LED display or LCD display capable of displaying graphical information and supporting an onscreen keypad function.

### EXAMPLE

### Temperature Sensor Circuit

Referring to FIG. 2, the circuit, according to a specific implementation of the present invention uses a thermistor 200, a resistor bridge 201 and an instrumental amplifier 202. By placing the resistor at one of the legs of the resistor bridge, any temperature changes at thermistor 200 unbalance the bridge. This in turn produces a voltage output proportional to the resistive change. The temperature of the airflow (from the nostril) is heated further using a PTC surface heater (400, illustrated in FIG. 4) positioned directly in line with the airflow towards the sensor 200. The changes are amplified to a working range of voltages and passed to an analogue to digital converter (ADC, not shown) to be sampled and digitalised. By using the potential divider to the references on the ADC, the voltage range can be reduced and the resolution of a binary bit change to further improve accuracy.

FIG. 3 shows a printed circuit board (PCB) design of the circuit of FIG. 2. The thermistor 200 is kept slightly away from the other components in order to prevent heating of the instrumental amplifier and the resistors. Any heating may cause drift in the component readings and tolerances.

Using a board size and design of FIG. 3, 0 to 3.3V proved to be a sensible reference voltage range for the ADC. No reference pin meant that the voltage dividers could be omitted and the board reduced to dimensions of 12 mm×40 mm allowing it to be hosted onto the opposite side of the heating tunnel (401, illustrated in FIG. 4). FIG. 5 is a further illustration of the board layout of the reduced component circuit.

The general mathematical elements of the electronic components were input into MATLAB (a numerical computing environment by Mathworks Inc.) using component and simulated values to produce digital values that were output to devices such as a personal computer (PC) (800, illustrated in FIG. 8). The circuit that was simulated was an ideal circuit that did not take into considerations tolerances, temperature changes due to self-heating components or drift due to applied voltages.

The code starts by allowing the user to input values for the resistance of the thermistor 200 and the gain of the instrumental amplifier 202. The code then determines the resistance of the thermistor at a specific temperature.

This is replicated in MATLAB for temperature values of interest. The airflow from the nasal area (or from the mouth) would generally be heated to around 35° C. with room temperature set at 25° C.

MATLAB then replicates the thermistor changing temperature based on a heated airflow from the nasal area hitting it. The thermistor 200, located at one leg of a resistor bridge, produces a positive change based on a decreasing resistance, the more positive the temperature sensed.

If the bridge has equal resistance over resistors 201 and the thermistor 200 the output will always be 0. If the thermistor were to decrease in temperature then the resistance would go positive and the result would be a negative voltage. This would causes problems when interfacing the output with the ADC. Accordingly a separate dual power supply was needed (illustrated in FIG. 8).

The resistor bridge then outputs a voltage based on the temperature change of thermistor 200 changing the bridge resistance. The output changes are relatively small, usually in the region of mV. Therefore, the instrumental amplifier 202 is used to accurately amplify the voltage output from the bridge. The magnitude of the amplification is set using a feedback resistance value.

Using MATLAB the amplification was limited to 2 or 5 times the input voltage to the instrumental amplifier 202 as a larger value would limit the temperature range that can be measured. Finally, the output from the instrumental amplifier 202 was required to be digitised. A MCB2300 evaluation board by Keil Elecktronik GmbH was used comprising a 10-bit analogue to digital convertor module 601 illustrated with reference to FIG. 6. The voltage reference range for the ADC 601 was set between 0 and 3.3 V. This difference is divided over 1024 steps of digital value. For this reason one increment in digital value is easily calculated. A digital value of 1 is a result of 3.22 mV being output from the instrumental amplifier 202.

### Modifying the Respiratory Airflow

In order to prevent the exhaled airflow 105 during respiration cooling over the short distance to the temperature sensor 200 it is heated by an air heater 407, illustrated in FIG. 4.

Heater 407 comprises a hollow metal tube 401 having a first open end 406 and a second open end 405 to allow airflow through the tube from end 406 to end 405. An aperture 404 is provided through the wall of the tube 401 extending substantially its full length. A PCT surface heating element 400 is positioned on top of the square cross section metal tube 401 to close aperture 404. The heating surface of element 400 is therefore exposed to the internal airflow conduit defined by the walls of the tube 401 between ends 406 and 405.

The PCB 402 illustrated in FIG. 5 is located external to the tube 401 and comprises the thermistor 200 mounted at one end so as to project into the region of tube end 405, such that resister 200 is positioned in the airflow path as the air exits tube 401 via open end 405. Tube 401 preferably comprises a high thermal conductivity metal so as to efficiently transfer heat from heating element 400 to the air flowing through tube 401 between ends 406 and 405. Suitable thermal insulation (not shown) may be provided around tube 401 and heating element 400 so as to prevent transmission of heat to the outer case 101 of the handheld device 100.

In use, the warm exhaled air 105 flows into the device 100 via funnel 102 and inlet port 107. The airflow then passes into metal tube 401 via open end 406 where it is heated by heating element 400 as it passes from end 406 to end 405. The heated air is then incident upon thermistor 200. The corresponding resistance change of thermistor 200 unbalances the bridge illustrated schematically in FIG. 2 to provide a voltage change. This voltage change is then amplified and converted to a digital signal for onward transmission to further electronic components for processing and ultimate output and display.

Referring to FIG. 6, the microcontroller evaluation board 605 incorporates a microcontroller and processor 602 used for testing and interfacing the thermistor circuit of FIG. 2 and for monitoring the respiration cycle. The microcontroller board 605 further comprises a processor 602, communication ports 600, an analogue digital convertor 601 and a potentiometer 604.

The microcontroller hosted by the MCB2388 incorporating the 10-bit ADC 601 further comprises elemental Input/Output (I/O) pins for introducing external analogue signal inputs, i.e. the output from the thermistor/Instrumental amplifier circuit of FIG. 2 and timer functions for controlling the I/O's. Other features of the Keil MCB2388 board include two push buttons, LED's, and an analogue piezo buzzer that fall in the I/O pin category.

The MCB2300 board connects the on-chip serial Universal Asynchronous Receiver/Transmitter (DART) to the MAX563 (IC2), which converts the logic signals to RS-232 voltage levels, therefore allowing serial communication with a PC 800 (referring to FIG. 8). The communication port 600 connector was wired to allow a board reset via the Data Terminal Ready (DTR) pin, and enable In-System Programming (ISP) via a Request to Send (RTS) pin. RS232 serial communication was selected to allow the capture and logging of data that in turn could be manipulated to allow evaluation and discussion of results.

The Keil MCB2300 has a number of pins available for I/O ports. Some are multiplexed and as such are not fixed to a peripheral on the board. Others, such as the piezo analogue buzzer, which is assigned an I/O pin as an output may not be used as a general purpose I/O port. Another of these assigned pins belongs to the potentiometer 604. The potentiometer 604 is assigned as an analogue input and uses pin 606. This pin 606 is connected via a jumper to the 10 kΩ potentiometer 604. The potentiometer (POT) varies the resistance between a 0-3.3V supply, the same voltage typical to the ADC 600 on the microcontroller 602.

By removing the jumper and connecting an analogue output to pin 606, this by-passes the potentiometer 604, but essentially performs the same task, i.e. digitising a varying analogue input to pin 606. Therefore, by connecting the output from the circuit of FIG. 2 digital out values were obtained.

The ADC converter module 601 in processors 602 has 8 input channels which allow conversion of an analogue input signal to a corresponding 10-bit digital number. This ADC 601 was used to convert the output from the instrumental amplifier 202 in the circuit in FIG. 2 to a corresponding digital value.

The ADC's digital values are then captured in real-time by LabVIEW and simultaneously output to a HyperTerminal session found on most PC's with a Microsoft operating system, for the purpose of data transfer and logging.

### System Software

A number of software applications were used within the present embodiment. LabVIEW utilises a high level coding to run the various peripherals on the MCB2388 board 605, in particular the LCP 2388 microcontroller 602. It was also used to produce graphical real-time analysis of the breathing monitor circuit illustrated in FIG. 2. HyperTerminal found on most Microsoft operating systems was used to receive data from the MCB2388 board 605 for the purpose of data logging and analysis.

### LabVIEW

LabVIEW by National Instruments, was used for test, measurement and control of the present electronics. It provides an interface with measurement and control hardware/circuitry, analysis of data and results sharing. It also allows the user to produce high-level language coding using a graphical interfacing. This coded program can then be compiled into machine language as part of the LabVIEW software. LabVIEW allows the user to view two different aspects of the coding interfaces. The first is the block diagram view where the coding based on high-level language is coded. The second is the front panel of the VI (Virtual Instrumentation). This is where the virtual instrumentation and any controls and indicators, which are interactive input and output terminals of the VI respectively, are built. Every controller or indicator on the front panel has a corresponding terminal on the block diagram interface. LabVIEW also contains programming concepts such as for loops and while loops to allow a program to run continuously until a stop condition occurs.

### Converting to Temperature

Within the While loop a first block is created to read the analogue input of the LCP2388 602. This corresponds to the pin 606 which will be the output from the instrumental amplifier 202 of the breathing monitor circuit of FIG. 2. Via the front panel user interface a ‘Graph Waveform Chart’ and a ‘thermometer’ indicator are placed. The corresponding blocks for the chart and thermometer are also placed on the block diagram interface. A read analogue input is firstly divided by a constant of 54.6, then the respective output from this block 700 has a constant of 22 added as illustrated in FIG. 7.

This provides conversion of a digital value between 0-1023 to its respective temperature in degrees Celsius. The conversion factors were calculated using the values obtained from the ideal circuit within the MATLAB simulation.

The input to the ADC 601 was between 0 and 3.3V and held by an ADC reference voltage. One digital increment is therefore equal to 0.0032 mV.

The MATLAB program (which replicated the outputs of an ideal circuit of FIG. 2), outputs the digital values at the corresponding temperatures (based on the thermistor R/T Curve). In particular, 35° C.=Digital Value of 710=0.0032V×710=2.272V; 22° C.=Digital Value of 147=0.0032V×0=0V. This is a difference of 2.272V−0V=2.272V, over a range of 13° C. This is a Digital Value difference of 710−0=710, over a range of 13° C. Therefore:

$$1 ° C . = \frac{2.272}{13} = 0.1747 V$$

$$\frac{1 ° C .}{Digital Increment Volt a ge Value} = \frac{0.1747}{0.0032} = 54.6 steps per 1 ° C .$$

Accordingly, for every time the digital value increments 56.3 the output has moved 1° C. in temperature. As the range calculated range started from 22° C. this needs to be added as a constant.

### Threshold and Threshold Limit

Ambient temperature may vary from room to room and the heated airflow through conduit 401 may cool more rapidly in a lower room temperature than it would in a room of higher temperature. A threshold input block and a threshold indicator 701 were added to accommodate this effect. The threshold input block allows the user to vary the threshold, i.e. the point at which a definite temperature change is registered and output light the indicator 701. According to further embodiments, the determination of the threshold value may be automated by the device via a temperature sensor incorporated at the device and suitable software.

### Breathing and Breathing Rates

One function of the breathing monitor device 100 is to monitor and count the number of respirations over a specific time interval. In turn, this value needs to be averaged over the time interval to produce a respiration rate, usually in cycles per minute. These features were first realised in C# programming. A single breath was registered when the threshold 701 passed on the rising edge, but not on the falling edge of a waveform. Another breath was not registered until the rising edge was detected once again. Another variable called ‘Numeric’ was created and declared and initialised to 1 outside the While loop and an additional variable called breaths was also created. It allows the output to be viewed in the front panel while creating a block in the block diagram to use as a variable. FIG. 7, block 702 illustrates how this was realised in LabVIEW using function blocks.

Code then utilised the output from the breaths variable to produce a breathing rate over one minute. Within the While loop a ‘Wait Until Next ms Multiple’ function block was placed. This waits until the value of the millisecond timer becomes a multiple of the specified millisecond multiple. It is generally used to synchronise activities and is called within loops to control the loop execution rate. A rate of 20 ms was applied based on the fastest operation placed upon a typical breathing monitor.

A small healthy child 106 may typically have a breathing rate between 20-60 breath cycles per minute and therefore a maximum of 1 cycle per second. In order to sample the respiration, the sampling theorem indicates that the sample rate should be at least twice the signal's highest frequency component. At 20 ms:

$$\frac{1 s}{20 ms} = \frac{1}{0.02} = 50 samples \text{/} sec$$

This provides an adequate sampling rate for capturing an accurate, waveform without an over exuberant demand on processing. To evaluate the number of samples this becomes over one minute:

$$60 \times \frac{1}{0.02} = \frac{60}{0.02} = 3000 samples$$

A new variable was created called ‘Numeric_{—}2’. The aim of this was to increment every time the While loop had finished an iteration loop every 20 ms. A ‘less than’ block in LabView placed ‘Numeric_{—}2’ in a condition to not carry out any function until the number of loops had surpassed 3000. The conditions on passing this count were placed in a case statement to pass the value of breaths to a new variable ‘breathing_rate’, before resetting ‘Numeric_{—}2 and breaths back to 0 to start the count again. The Breathing Rate block also appears in the front panel as an indicator referring to FIG. 7.

### Capturing Data

In order to capture the data and send it to a HyperTerminal session, the serial connection (RS-232) was used. Using Lab VIEW, the first block as part of the output to the HyperTerminal converts the temperature modified output from the ADC, to a fractional numerical value illustrated as 704. The output of the Number to fractional string block becomes an F-fractional string. This was then fed into a ‘string concatenate’ block. This concatenates input strings and one dimensional array of strings into a single output string (704). Three inputs were fed in order into the concatenate block.

The concatenated string was then fed into a ‘serial port write block’ which writes data strings to serial ports that were specified by the port number input to the block. The port number was indicated by placing a corresponding constant number to the port number input of the block (705).

All that remains is the ‘serial port Init’ that initializes the selected corresponding serial port to the settings specified. This block was tied to the same constant block as the ‘serial port write block’ 705.

### HyperTerminal

Referring to FIG. 8, HyperTerminal's application in the present embodiment was to allow the sampled digital values to display on a connecting PC or Laptop 800. The PC 800 was connected to the MCB2388 evaluation board 605 via an RS-232 serial communication port. The configurable parameters have to match at either end of the communication terminals and mismatches in baud rate for example will produce random or no characters on the displaying terminal. HyperTerminal was used purely for capturing and logging data to be analysed and evaluated further. The actual real time breathing/respiration rate was captured locally by the virtual instrumentation.

The setup for testing comprised of a varying dual power supply 801, the MCB2388 microcontroller evaluation board 605, the breathing monitor sensor and heater circuit illustrated in FIG. 2, a laptop 800 running LabVIEW 2009 ARM edition and a PC (not shown) running HyperTerminal. Although the latter two could be run on the same PC or laptop, for ease of capturing the data in testing, they were separated.

The Dual power supply 801 was setup with each supply terminal set to 5V, needed as a regulated power supply for the PCB board 402 and the Instrumental amplifier 202. The Instrumental amplifier 202 needed ±5V supply rails in order to operate, so inversion of one of the supplies was needed. To achieve the negative supply terminal of the left right hand supply was connected with a lead to the positive terminal of the left hand supply. Then, this was connected to ground to stop the ±5V ‘floating’ and give it a point of the ground reference.

The right hand positive supply terminal, which became the +5V supply was then connected to the positive supply rail terminal on the PCB board 402 mounted on the side of the heated air conduit 401. The negative supply terminal of the left hand power supply, now the −5V supply was connected to the −5V supply rail on the breathing Monitor PCB Board circuit of FIG. 3.

The PTC Heater element 407 encased in a PCB Material to act as a heat-sink for convecting heat was wired to a mains supply via a fused 3 Amp plug as specified by the manufacturer. The whole breathing monitor was clamped in place on an arm in the air flow of subjects who were lay horizontally.

For real time data capture a USB connection to a JTAG Debugger on the MCB2388 board 605 was connected to a laptop running LabVIEW Virtual Instrumentation on Windows XP. Data were captured using a serial lead from the communication port 600 to a PC running HyperTerminal.

### Test Session

The respiration rates of two voluntary subjects (A and B) of different ages, heights and physical conditions were determined using the apparatus described herein.

The device 100 was placed in the airflow of both subjects 10 cm from the nasal cavity and its respiration airflow. The airflow direction could be checked quite quickly by placing the back of the hand about the same distance away in front of the nasal area. Any sudden fine tuning and adjustments could be made by using the virtual instrumentation in LabVIEW.

Referring to FIG. 9, the user interface displays the respiration cycle as a waveform having temperature for vertical axis over time (horizontal axis) via display 901. An indicator 902 displays the temperature at thermistor 200. The threshold temperature limit 701 ensures a cycle is recorded only where the temperature of the thermistor rises above the preset value. The results of the waveform generation can then be used to calculate the breathing rate 900.

Both subjects were tested over a 4-minute period once a good respiratory response was achieved. Once a good respiratory response was in place, the temperatures being achieved allowed the threshold to be set on the front panel of the virtual instrumentation. The threshold in each case was set to 35° C. FIG. 10 shows a 30 second sample of the captured data by the HyperTerminal session. The captured data 1000 was plotted against its relevant time interval (which was every 0.02). Results for the breathing rate were recorded by hand and detailed in table 1, as they only changed at every minute interval.

TABLE 1
Subject A - Breathing/Respiratory Rate

| Time interval (in | Respiration Rate |
|---|---|
| minutes) | (in cycles per minute) |
| 1 | 11 |
| 2 | 13 |
| 3 | 12 |
| 4 | 11 |

Using the same experimental setup, in the same environment, subject B was tested over a 4-minute period. FIG. 11 and table 2 display the captured data and the respiratory rate using the virtual instrumentation.

TABLE 2
Subject B - Breathing/Respiratory Rate

| Time interval (in | Respiration Rate |
|---|---|
| minutes) | (in cycles per minute) |
| 1 | 11 |
| 2 | 10 |
| 3 | 10 |
| 4 | 11 |

The results show an excellent measured respiratory signal and equally respectable respiration rate for each of the subjects. A respiratory waveform is clearly visible in each case. Referring to FIG. 12, the rising edge 1200 of the waveforms is due to the expired air from the subjects, whereas the peak, comprising of the change from the rising edge to the falling edge of the waveform is considered the expiratory pause (1201). The falling edge represents the point at which the subjects are inspiring air into the lungs and hence no heated airflow is pushed towards the sensor circuit (1202). The trough of the waveform just before the waveform returns to a rising edge is the inhalation pause (1203). Both the peak and the trough of the waveform are considered transition points at which no airflow flows and collectively this defines one complete breathing or respiratory cycle.

Subject A has the more consistent respiratory signal that varies from around 34° C. to 39° C., a difference of 5° C. overall. It remains reasonably consistent in one case reaching a peak 1001 of 40° C. As the threshold within the monitoring software was set to 35° C., slight variance, of less than 1° C. did not affect the respiratory rate. Subject B has a slightly less consistent respiratory signal that does ‘wander’ slightly more than subject A. The waveform is not as differential as subject A and varies from roughly 34° C. to 36° C., a difference of 2° C. The waveform amplitude of subject B is 3° C. less than subject A. This difference in amplitude may have clinical significance.

The respiration rate was also calculated by taking the time of one cycle (peak to peak) from the waveforms 1000 and 1100 and dividing it by 60 seconds. Using the data of FIGS. 10 and 11, table 3 shows the calculated respiratory waveforms of both subjects.

TABLE 3
Calculated Respiration Rate

| | Peak A (in | Peak B (in | Respiration Rate in |
|---|---|---|---|
| Subject | seconds) | seconds) | Cycles per minute |
| A | 2.2 | 7.7 | $\frac{60}{7.7 - 2.2} = 10.9$ |
| B | 67.9 | 74.2 | $\frac{60}{74.1 - 67.9} = 9.7$ |

According to further specific implementations, the PCT heating element 200 may be supplied by a DC supply rather than an AC supply enabling the entire circuit to be driven by a single DC battery. Furthermore, the board may comprise suitable ports or docking stations to allow the battery to be recharged. Any such ports may enable connection for data download or upload with a PC, server, network or the internet. According to a specific embodiment, where multiple circuit boards are implemented, a battery may be provided for each respective board so as to provide independent power supply.

According to the embodiment described with reference to FIGS. 1 to 12, the system uses virtual instrumentation to take a breath rate based on a user defined threshold using the ADC 601 on board 605. This board could be omitted and replaced by a comparator circuit. This further embodiment could register a change in the comparator circuit and produce a binary output for subsequent processing. When comparator is employed, the need for analogue to digital converter is eliminated.

According to a further implementation, a moving average could be developed into the software to address any ‘wandering’ temperatures associated with the temperature verses time waveforms of FIGS. 10 and 11 and to enable more accurate determination of breaths or cycles per minute. In this mode, a window with a predefined length of time is selected. The respiration rate within the window is determined and then the window is shifted in time by a suitable amount to repeat the process. The respiration signal can also be suitably filtered either by an integrated analogue filter or it can be filtered digitally once recorded. This filtering removes any unwanted frequency component of the signal.

1. A respiration monitoring device comprising:
an airflow inlet port to allow a flow of exhaled air from a human or an animal into the device;
a temperature modifier to receive the flow of the exhaled air via the airflow inlet port and to heat the exhaled air;
an electronic temperature sensor to detect a change in temperature of the exhaled air within the device; and
an airflow tunnel configured to direct the flow of the exhaled air from the airflow inlet port to the temperature sensor, the temperature modifier positioned at and external to the tunnel so as to heat the exhaled air as the exhaled air flows through the tunnel;
wherein the temperature sensor is positioned external to an open airflow exit end of the tunnel in an airflow path exiting the tunnel downstream of the temperature modifier to receive the heated flow of the exhaled air from the tunnel.

2. The device as claimed in claim 1 wherein the electronic temperature sensor comprises a thermistor.

3. The device as claimed in claim 2 wherein the thermistor is an NTC or PTC thermistor.

4. The device as claimed in claim 1 wherein the temperature modifier comprises a PTC heater.

5. The device as claimed in claim 1 further comprising a funnel extending from the airflow inlet port to direct the flow of the exhaled air into the airflow inlet port.

6. The device as claimed in claim 5 further comprising means to releasably attach the funnel to the device.

7. The device as claimed in claim 1 further comprising an airflow outlet port to allow the flow of the exhaled air to exit the device once the flow of the exhaled air has passed the temperature sensor.

8. The device as claimed in claim 1 further comprising an electronic display screen.

9. The device as claimed in claim 8 wherein the display screen comprises an LCD display.

10. The device as claimed in claim 8 wherein the display screen comprises a touch screen device.

11. The device as claimed in claim 1 further comprising a microchip.

12. The device as claimed in claim 1 further comprising an electronic memory for data storage.

13. The device as claimed in claim 1 further comprising electronic communication means to enable wired or wireless information transfer from the device.

14. The device as claimed in claim 1 further comprising an analogue to digital convertor.

15. The device as claimed in claim 1 further comprising a microprocessor.

16. The device as claimed in claim 1 further comprising at least one battery power source.

17. A method of monitoring respiration of a human or an animal comprising:
receiving a flow of exhaled air via an airflow inlet port;
directing the flow of exhaled air within the device through a tunnel;
heating the flow of the exhaled air received from the air flow inlet port within the tunnel using a temperature modifier, the temperature modifier positioned at and external to the tunnel so as to heat the exhaled air as the exhaled air flows through the tunnel; and
detecting a change in temperature of the exhaled air within the device using an electronic temperature sensor positioned external to an open airflow end of the tunnel at one end of the tunnel in an airflow path exiting the tunnel downstream of the temperature modifier as the heated exhaled air flows from the open end of the tunnel.

18. The method as claimed in claim 17 further comprising directing the flow of the exhaled air from the human or the animal into the device using a funnel attached to the device.

19. The method as claimed in claim 17 further comprising processing data generated from the temperature sensor.

20. The method as claimed in claim 19 further comprising outputting the processed data at a visual display.
